(19) United States
(12) Patent Application Publication
(10) Pub. No.: US 2014/0199240 A1
(43) Pub. Date: Jul. 17, 2014

(54) ANTICANCER AGENT

(71) Applicants: Jan Rijn Zeevaart, Brits District (ZA); Xavier Yangkou Mbianda, Doornfontein (ZA); Zoltan Szucs, Brits District (ZA)

(72) Inventors: Jan Rijn Zeevaart, Brits District (ZA); Xavier Yangkou Mbianda, Doornfontein (ZA); Zoltan Szucs, Brits District (ZA)

(73) Assignees: UNIVERSITY OF JOHANNESBURG, Auckland Park, Johannesburg (ZA); THE SOUTH AFRICAN NUCLEAR ENERGY CORPORATION LIMITED, Brits District, Pelindaba (ZA)

(21) Appl. No.: 14/130,344

(22) PCT Filed: Jul. 2, 2012
(86) PCT No.: PCT/IB2012/053348

§ 371 Date: Feb. 7, 2014

Related U.S. Application Data
(60) Provisional application No. 61/503,703, filed on Jul. 1, 2011.

(51) Int. Cl. A61K 51/04

(52) U.S. Cl. CPC A61K 51/0497
USPC 424/1.77; 562/22; 562/13

(57) ABSTRACT

The invention relates to a method for preparing a bisphosphonate covalently bonded to a nanostructure. This invention also relates to a bisphosphonate having incorporated therein a radioisotope selected from ³²p or ³³P, preferably ^{33p}, wherein the bisphosphonate is covalently bonded to a nanostructure directly or by way of a linker, and to the use thereof in a method of treating calcific tumours in a patient.

### BACKGROUND OF THE INVENTION

This invention relates to the treatment of secondary, metastatic bone cancer and to anticancer agents for such treatment.

Statistics from cancer research show that most of the different forms of primary cancer preferentially metastasize to the skeletal system or more specifically to bone tissue, forming delocalised secondary or metastatic bone cancer; a painful skeletal situation. Chemotherapy has been established as the most effective form of treatment, since the drugs administered can reach most areas where the metastatic cancerous cells have relocated. Numerous drugs have been developed for the treatment of bone lesions. However, owing to their general low molecular weight, these drugs have a limited success because of premature excretion or renal clearance. Furthermore, the low molecular weight drugs typically lack the ability to discriminate between normal tissue and tumour tissue. Together with a tendency to administer the drugs in large doses in a bid to overcome renal clearance the excessive administration of the drugs frequently lead to systemic toxicity.

The development of more efficient drugs in terms of their toxicity, half-life, bio-distribution and degradation has produced an agent with great promises, namely the family of bisphosphonates or diphosphonates. This is a unique family of phosphorous-based compounds, analogues to pyrophosphates, characterized by two C—P bonds located on the same carbon. Earlier uses of bisphosphonates were mainly industrial as corrosion inhibitors, complexing agents in textile, fertilizer and oil industries. Polyphosphates are known to be able to inhibit crystallization of calcium salts, thus acting as water softeners. Investigations of bisphosphonates for clinical uses found that pyrophosphates have an ability to prevent hardening of soft tissue (calcification) by binding onto newly forming bone mineral (hydroxyapatite).

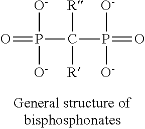

Bisphosphonates likewise have the ability to accumulate at the sites of bone metastasis because of their very high affinity for bone mineral undergoing renewal. Their structure fits their function in the sense that the hydroxyl group at the R position together with the two phosphonate groups (often referred to as a hook) has a high affinity for bone mineral. The R′ side chain determines chemical properties, biological activity as well as the strength of the bisphosphonates. Thus, they are able to inhibit tumour induced bone resorption, correct hypercalcemia, reduce pain, prevent new osteolytic lesions and can prevent fracture occurrence. These compounds may therefore be used as targeting molecules or vehicles for anticancer activity with a high specificity for the site of bone metastasis, which may afford reduced dosing and, thus, reduced systemic toxicity.

Various forms of bisphosphonates have been developed mainly as palliative drugs. However, as with other low molecular weight drugs they have the same inescapable shortcoming of excessive renal clearance before reaching their targeted tumour lesions. In addition, bisphosphonates have a poor bioavailability. Only about 3-7% of the bisphosphonates are systemically available. As renal excretion is the only route for elimination, the efficiency of bisphosphonates in treating bone related illnesses depends on ensuring that the amount of excreted bisphosphonates is reduced.

However, at best these drugs can only retard the degrading effect of cancerous tumour cells on the surrounding healthy tissue, but do not afford a means of killing or reversing the proliferation of the tumour cells.

It is an object of the present invention to provide improved bisphosphonate anti-cancer agents.

### SUMMARY OF THE INVENTION

According to a first aspect of the invention, there is provided a method for producing a bisphosphonate; the method including the steps of:
providing a compound or nanostructure having carboxylic acid functional group/s; and
reacting the compound or nanostructure with phosphoric acid and a chlorinating agent such as phosphorous trichloride or phosphorous pentachloride or other chlorinating agent such as the oxychlorides (e.g. thionyl chloride (SOCl₂) or phosphorous oxy trichloride (POCl₃), preferably phosphorous trichloride, in an organic solvent such as methane sulphonic acid.

Preferably, the bisphosphonate is a radiolabelled bisphosphonate having incorporated therein a radioisotope selected from ³²P or ³³P, preferably ³³P; and the phosphoric acid contains a radioisotope selected from ³²P or ³³P, preferably ³³P.

Additives that may be added are hypophosphorous acid (H₃PO₂) or ethanedinitrile (cyanogen—C₂N₂).

The compound may be a carboxylic acid which may be selected from single chained or branched hydrocarbons that may contain amine groups (secondary or tertiary), preferably amino-propanoic acid.

Typically, the carboxylic acid has the structure R(CH₂)ₙCOOH, wherein:
R═CH₃ or NH₂; and
n=1, 2 or 7.

In a preferred embodiment of the invention, the nanostructure is a carbon nanostructure and the method produces a radiolabelled bisphosphonate which is covalently bonded to a carbon nanostructure directly or through a linker, as described above.

Carbon nanostructures which exhibit carboxylic acid functional group/s may be produced by applying defect site chemistry to covalently bond carboxylic acid functional group/s to carbon nanostructures, wherein defects on the carbon nanostructures are induced by oxidation with a strong acid such as nitric acid.

According to a second aspect of the invention there is provided a bisphosphonate having incorporated therein a radioisotope selected from ³²P or ³³P, preferably ³³P, wherein the bisphosphonate is covalently bonded to a nanostructure directly or by way of a linker.

A “linker” is a group, such as alkyl containing from 1 to about 10 carbon atoms, alkenyl containing from 2 to about 10 carbon atoms, amino or substituted amino, which is covalently bonded to the bisphosphonate and to the nanostructure, and which links the two by way of covalent bonding.

Preferably, the bisphosphonate covalently bonded to the nanostructure has a molecular weight of greater than about 40 kDa and less than about 400 kDa or in terms of a nominal diameter or principle dimension between about 5 nm to about 500 nm, the latter in case of a substantially tubular structure, preferably about 40 kDa to about 120 kDa, most preferably about 60 kDa to about 100 kDa, typically about 80 kDa, or about 20 nm to 120 nm, typically about 50 nm to 100 nm, depending on the form of nanostructure taken.

The bisphosphonate may have the general structure:

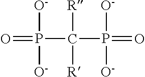

where:

R′ is hydrogen, alkyl containing from 1 to about 20 carbon atoms, alkenyl containing from 2 to about 20 carbon atoms, aryl, phenylethenyl, benzyl, halogen, hydroxyl, amino, substituted amino, —CH₂COOH, —CH₂PO₃H₂, —CH(PO₃H₂)(OH), or —CH₂C(PO₃—H where n is 1 to 15; and

R″ is a nanostructure, which may be linked to the bisphosphonate by a linker such as alkyl containing from 1 to about 10 carbon atoms, alkenyl containing from 2 to about 10 carbon atoms, amino or substituted amino.

Preferably, R′ is hydroxyl.

The nanostructure is preferably a carbon nanostructure, most preferably a carbon nanotube.

The carbon nanotube may be single-walled or multi-walled, preferably multi-walled, most preferably double-walled.

The single-walled nanotube may have a diameter of 0.4 to 5 nm, preferably 0.5 to 1 nm.

The double-walled nanotube may have a diameter of 4.5 to 100 nm, preferably about 5.0 nm.

The nanostructure can also be a nanosphere, preferably a carbon nanosphere with a diameter of about 5 to 20, typically about 12 nm or molecular weight of about 50 to 100 kDa, typically about 80 kDa.

According to a third aspect of the invention, there is provided a method treating calcific tumours by administering a radiolabelled bisphosphonate which is covalently bonded to a nanostructure directly or through a linker, as defined above, to a patient in need thereof. The invention also relates to a radiolabelled bisphosphonate which is covalently bonded to a nanostructure directly or through a linker, as defined above for use in the treatment of calcific tumours, as well as the use of a radiolabelled bisphosphonate which is covalently bonded to a nanostructure directly or through a linker, as defined in a method of manufacturing a medicament or use in the treatment of calcific tumours in a patient in need thereof.

### BRIEF DESCRIPTION OF THE DRAWINGS

FIG. 1 is a Raman spectra of pristine double walled carbon nanotubes showing disorder band at 1350 cm⁻¹ and the tangential band at 1578 cm⁻¹;

FIG. 2 is a Raman spectra for oxidised double walled carbon nanotubes showing a shift of the wave number to the right and a decrease of the I_{D}/I_{G} ratio corresponding to the increase in tangential mode (TM); and

FIG. 3 is Raman spectra of bisphosphonates compounded carbon nanotubes.

### DESCRIPTION OF PREFERRED EMBODIMENTS

According to a first aspect of the invention, bisphosphonates are synthesized as a family of therapeutic drugs. In one embodiment of this aspect of the invention, a non-radioactive (cold) bisphosphonic acid may be prepared by reacting a carboxylic acid with phosphoric acid and phosphorus trichloride, in methane sulphonic acid (MSA) as a solvent as exemplified in Scheme 1.

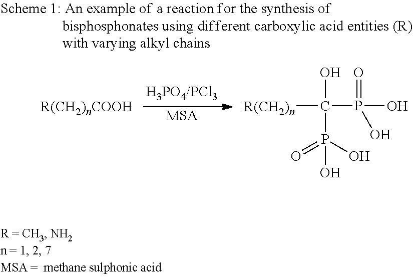

Using the same reaction scheme biocompatible bisphosphonic esters may be synthesized by neutralising the corresponding acid with sodium hydroxide (NaOH). For instance, CH₃(CH₂)₅COH(PO(OH)₂)(PO(OH)Na) (1-hydroxy heptilydene bisphosphonic acid monosodium salt) or H₂N(CH₂)₂COH(PO(OH)₂)(PO(OH)Na) (1-hydroxy-3-amino-propilydene diphosphonic acid monosodium salt or pamidronate).

In a preferred embodiment of this aspect of the invention, radioactive ³²P/³³P-hydroxy heptyl bisphosphonates may be synthesized by substituting the H₃PO₄ with a radioactive H₃PO₄ as exemplified in Scheme 2. In this instance the amount of reactants and products will be at a much smaller scale, typically micro scale and in appropriately protected areas, such as a hot cell or suitable glove box, since it involves radioactive materials. Thus, the active bisphosphonate group may be attached to a alkane chain of a selected length as the drug delivery system or carrier.

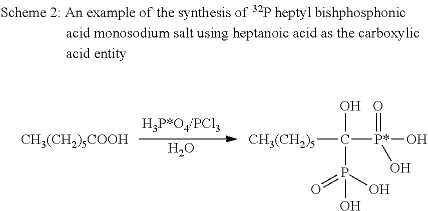

In another embodiment of the invention macromolecular drug carriers in the form of nanostructures are covalently bonded to the anticancer agent bisphosphonate group in order to utilise the EPR effect. The inventors have found that amongst others double wall carbon nanotubes are highly suitable candidates for this purpose.

Double wall carbon nanotubes may be synthesized by the catalytic chemical vapour deposition method (CCVD). The impurities from this process like unreacted catalyst and amorphous carbon are removed by air oxidation, acid treatment, sonication and micro filtration. The purified, pristine carbon nanotubes are then chemically modified by functionalisation to render the nanotubes solubilised, biocompatible and less toxic, facilitating their use as drug delivery vehicles. By applying defect site chemistry functional groups are covalently attached onto the skeleton of the carbon nanotubes. Defects on the carbon nanotube side walls are induced by oxidation with a strong acid such as nitric acid. This is described in more detail in the following steps.

Removal of amorphous carbon by gas phase oxidation of the double walled carbon nanotubes as produced from the CCVD method was achieved by heating the carbon nanotubes in a furnace at 350° C. for about 30 minutes. This was done in an open quartz tube (without gas connections) to allow for free flow of air.

Dissolving of the catalyst residues was achieved by adding a hydrochloric acid solution of about 1M (vol/vol) to the carbon nanotubes in a beaker. The suspension was then stirred and left to settle at room temperature for about 15 minutes. The beaker was then covered with parafilm. The suspension was subjected to sonication for 30 minutes to disperse the clustered tubes and other smaller sized particles so as to expose the individual tubes to adequate acid treatment and then left to settle once more before filtration in a microfiltration apparatus using Teflon PTFE with a pore size of 0.47 μm. To prevent acid corrosion of the filter paper, 150 ml deionised water was added to the solution and further washed until neutral. The pH was monitored by using universal indicator papers.

In preparing the carbon nanotubes for covalent bonding with the bisphosphonates, they were modified or functionalised by strong acid oxidation. This resulted in hole or defect formation on the side wall of the carbon nanotubes as well as cap removal (serving as a further purification step), thus, forming carboxyl functional groups such as carboxylic acid by oxidation of the open ended carbon atoms, as illustrated in Scheme 3. The resulting oxidised carbon nanotubes were then again washed and neutralized by microfiltration.

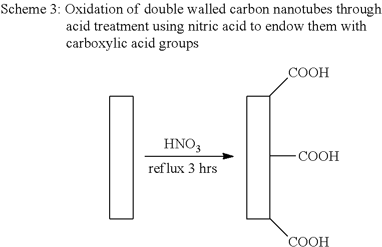

The oxidised (functionalised) carbon nanotubes may be covalently bonded onto bisphosphonates by reacting the induced carboxylic acid functional groups with phosphoric acid and phosphorus trichloride in MSA as a solvent, similar to the method described in Scheme 1. This reaction can be summarized as shown in Scheme 4,

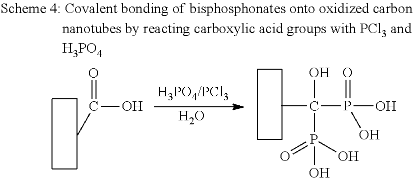

Synthesis of ³²P labelled covalently bonded bisphosphonate carbon nanotubes may be attained by following the same route as in Scheme 2 by performing the reaction with ³²P labelled H₃PO₄ instead of cold H₃PO₄ (see Scheme 5).

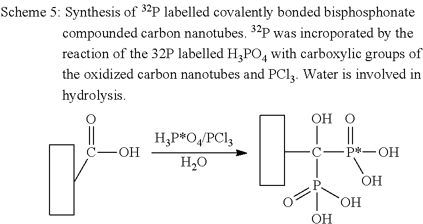

According to a second aspect of the invention there is provided a dual-purpose anticancer agent in the form of a radiolabelled bisphosphonate which does not only treat the damaged bone tissue and thus relieves the pain associated with metastatic bone cancers, but will also destroy at least some of the malignant tumour cells. The selectivity and target site retention of the anti-cancer agent is enhanced by increasing its molecular weight by covalent bonding to a carbon nanostructure. A second aspect of the invention relates to a method of synthesising such a dual purpose anticancer agent. A third aspect of the invention relates to a method or procedure of administering the anticancer agent to a human body system with high specificity and low systemic toxicity.

The radiolabelled bisphosphonate of the present invention combines the beneficial properties of bisphosphonates that are well known as palliative cancer drugs with the ability to destroy malignant tumour cells and adjacent osteoclasts. The specificity of the radiolabelled bisphosphonate is improved by covalently bonding it onto a carbon nanostructure to increase its molecular weight to greater than about 5 nm or 40 kDa which is above the threshold of renal clearance and is done in order to passively and preferentially accumulate in the tumour tissue due to the EPR (Enhanced permeability and retention) effect, exploiting the increased wall permeability of the dilated blood vessel system of cancerous tumors and to have enhanced specificity and bioavailability. The carbon nano structure is an ideal delivery system because it can accommodate a large number of drug anchoring sites, which will facilitate cell entry via cell membranes, and has the ability to control drug concentration as well as enhancing drug supply through slow drug release from the drug-delivery system anchoring sites/compounds. On the other hand, these particles should not exceed 200 nm to avoid removal as foreign bodies by macrophages.

Another effect that is exploited is the phenomenon of enhanced permeability retention (EPR) effect whereby macromolecules are preferentially trapped and retained within tumour tissue. Normal tissue transport of substances for the livelihood of the cells is across capillaries, which allows transport of smaller molecules rapidly without restriction, whereas macromolecules are unable to cross the capillaries and are instead removed via bulk fluid phase transfer (extravasation or rapid wash out). Characteristics of tumour tissue that enhance the retention of macromolecules include high vascular density. This results from the elevated nutritional needs of the tumour tissue compared to normal tissue. There is also a tendency for the over production of vascular mediators that facilitate extravasation of macromolecules from the blood plasma to the tumour tissues. The abnormal structure of the tumour vessels that include the stretching of the vessels as well as a weakened lymphatic clearance of macromolecules from the interstitial tissue also enhances the retention of macromolecules for a prolonged period of time.

On the other hand, lower molecular weight compounds such as unmodified bisphosphonates are returned to the circulating blood by diffusion, compounding to the negative effect of renal clearance of these species. Hence, increasing the molecular weight of the bisphosphonates by covalently bonding them onto an appropriate delivery system with a total molecular weight beyond the renal clearance threshold, further exploits the EPR effect to retain them in the tumour tissues for a prolonged period of time. This will allow greater interaction of the bisphosphonates with the tumour lesions for more effective treatment and simultaneously reduce systemic toxicity associated with large doses.

In radiotherapy, high energy radiation is used to destroy or kill cancer cells. The radioactive source could either be internal or external. Despite its capability to kill some cancer cells, radiation exposure of healthy tissue can damage DNA, resulting in secondary cancer. This is an adverse effect more often associated with external radiotherapy, where the radiation dose is uncontrolled and non-specific. Particularly in treating secondary bone cancer, external radiotherapy is not recommended due to the fact that tumours usually spread throughout the skeletal system.

Thus, according to this invention there is provided an anticancer drug and a method of producing same, which combines this anticancer activity with the efficiency of macromolecule drug carriers, to provide covalently bonded bisphosphonate carbon nanotubes radio labelled with ³²P or ³³P that have the potential of offering an excellent dual purpose therapeutic radiopharmaceutical that will enhance or supplement the tumour lesions treatment activity of the bisphosphonates. High and low energy betas (respectively) from the radioactive source are at the same time able to preferentially destroy the cancer cells. Through the EPR effect ³²P or ³³P labelled, covalently bonded, bisphosphonate carbon nanotubes may take advantage of the metabolic pathways and characteristics of tumour lesions, resulting in an increased bisphosphonates' effectiveness and reduced systemic toxicity as there will be a sustained release of the radiation energy within the tumour lesions as well as a prolonged exposure of the lesions to the drug (bisphosphonate), avoiding the need for continuous or prolonged administration of the drug.

A third aspect of the invention relates to a method or procedure of administering the anticancer agent to a human body system with high specificity and low systemic toxicity. Radiolabelled bisphosphonates having incorporated therein a radioisotope selected from ³²P or ³³P, preferably ³³P, covalently bonded to a nanostructure as described above may be used to treat calcified tumours such as bone metastasis. The anticancer agent may be formulated in a saline solution which may be administered to a patient in an injection or infusion.

The invention will now be described in more detail with reference to the following non-limiting Examples:

The experimental procedures in the following examples made use of characterization techniques that include the SEM, TEM, EDX, TGA, SXPS, HPLC and the NMR spectroscopy. The LSC was used to confirm the successful radio labelling of the covalently bonded bisphosphonate carbon nanotubes.

### Example 1

### Synthesis of 1-hydroxy heptyl bisphosphonic acid monosodium salt

1-hydroxy heptyl bisphosphonic acid monosodium salt was prepared by refluxing a mixture of 6.74 ml heptanoic acid, 4.6 g phosphoric acid, 40 ml methane sulphonic acid under an inert atmosphere according to Scheme 1. The mixture was heated at 65° C. as 8.25 ml of phosphorus trichloride was added with the mixture maintained at 65° C. and refluxed for 16 hours. The mixture was then cooled to 5° C. with 200 ml of deionized water added to the colourless solution which was left to reflux for 5 hours. The pH of the solution was adjusted to 4.3 through the addition of sodium hydroxide which resulted in the formation of a white suspension which was the expected product. The 1-hydroxy heptanoic bisphosphonic monosodium salt (product) was collected by filtration, washed with deionized water and ethanol and then air dried. The product analysis was as follows:

Yield: 9.60 g (73%); Mp=240° C.

IR/KBr (cm⁻¹): 3426 (O—H), 2935 (C—H), 1131 (P═O), 1027 (P—O), 920 (C—C)

¹H NMR/D₂O (ppm): δ 0.74 (t, CH₃CH₂, 3H, H-7 ³J_{H—H}=2.4), δ 1.17-1.18 (m, CH₃CH₂(CH₂)₃, 6H, H-6, H-5, H-4), δ 1.43-1.48 (m, CH₃CH₂CH₂—, 2H, H-3) δ 1.76-1.88 (m, CH₃CH₂(CH₂)₃CH₂, 2H, H-2)

³¹P NMR/D₂O (ppm): δ (18.62)

¹³C NMR/D₂O (ppm): δ 74.35 (t, ²J_{CP}=151), δ 38.48, δ 33.79, δ 30.95, δ 29.58, δ 23.72, δ 22.22

### Example 2

### Synthesis of 1-amino-3-hydroxyethyledene bisphosphonic acid monosodium salt (pamidronate)

Approximately 4.2 g alanine, 4.7 g phosphoric acid and 20 ml methane sulphonic acid were added into a flask that had been flushed with argon. The mixture was heated to 65° C. before phosphorous trichloride was added drop wise. The reaction mixture was left for reflux for 16 hours while maintaining the heat at 65° C. About 200 ml of deionised water was added into the reaction mixture for hydrolysis. The mixture was then refluxed for 5 hours. Sodium hydroxide was used to adjust the pH of the solution to 4.0. The solution was left to settle for 2 hours. A resulting white precipitate was filtrated and washed through a PTFE filter paper as in Example 1. The product analysis was as follows:

Yield: 7.70 g (55%); Mp=275° C.

IR/KBr, cm⁻¹: 1191 (P═O), 1049 (P—O)

¹H NMR/D₂O (ppm): δ 3.02 (t, NH₂CH₂ ³J_{H—H}=6.0, 2H), δ 1.96 (m, NH₂CH₂CH₂, 2H)

³¹P NMR, D₂O (ppm): δ (19.53)

¹³C NMR, D₂O/MeOD (ppm): δ 74.67 (t, ²J_{C—P}=137), δ 36.17, δ 32.80 (t, ²J_{C—P}=7.6)

### Example 3

### Synthesis of radioactive ³²P-hydroxy-heptyl bisphosphonates

For safe and efficient practice of nuclear medicine the procedures to introduce the radioactive agent into the carrier molecule should preferentially be performed at a micro scale. The experimental procedure for synthesizing ³²P-hydroxy-heptyl bisphosphonates according to Scheme 2 was similar to that which was used for the synthesis of normal (cold) hydroxy-heptyl bisphosphonates (Scheme 1, Example 2), with the exception that the H₃PO₄ was substituted with a radioactive H₃PO₄.

It should be noted that the amount of radioactive H₃PO₄ incorporated is in the order of 1 μg due to its high specific activity. The above reaction can be carried out with or without non-radioactive H₃PO₄ added (referred to as a carrier) to the level indicated in example 1. In other words, ³²P or ³³P can be added to any level of radioactivity as required.

For calibration purposes carrier added samples were prepared as standards by adding non radioactive H₃PO₄ to the radioactive sample in a solution state. The solution state ensures that the chemical stability of the radioactive ions is maintained, thus avoiding the formation of undesired complexes or adsorption of the ions onto the walls of the containers. Thus, a standard, carrier added radioactive bisphosphonate was prepared as follows:

³²P-hydroxy-heptyl bisphosphonates were synthesized by adding 100 μL ³²P labelled phosphoric acid (H₃PO₄) into a 10 mL vial then dried by blowing argon over the vial for 30 minutes. Then 13 μL H₃PO₄, 28 μL heptanoic acid, 100 μL methane sulphonic acid (as a solvent) were added into the vial. An inert atmosphere was achieved by flushing the reaction mixture with argon. This step was followed by the immediate addition of 44 μL PCI₃ to minimize PCI₃s' contact with the atmosphere which would otherwise lead to its hydrolysis. The mixture was heated to 65° C. in an oil bath on an electric plate for 20 hours after which 375 μL cold water was added for hydrolysis. The mixture was heated again to 65° C. for 5 hours. The solution's pH was adjusted to 4.0 by adding 125 μL of NaOH.

Similarly, carrier free ³²P-hydroxy-heptyl bisphosphonates were synthesized by adding instead 20 μL of ³²P labelled phosphoric acid (H₃PO₄) and no cold H₃PO₄. The rest of the procedure progressed as for the carrier added bisphosphonates.

Yield: 91% of the ³²P labelled phosphoric acid reacted.

### Example 4

### Synthesis of Double Wall Carbon Nanotubes

Double wall carbon nanotubes were synthesized by the catalytic chemical vapour deposition method (CCVD). A catalyst for carbon nanotubes synthesis was first prepared by dissolving 23.1 g magnesium nitrate hexahydrate, Mg(NO₃)₂.6H₂O, 2.9 g cobalt nitrate hexahydrate, Co(NO₃)₂.6H₂O, 30.9 g ammonium molybdate, (NH₄)₆Mo₇O.4H₂O, 76.9 g citric acid, C₆H₈O₇ in deionised water. The solution was heated on an electric plate for about 45 minutes (until most of the water had evaporated, leaving a thick paste). The paste was heated in an oven for 30 minutes at 550° C. The powder (catalyst of about 2 g) produced was heated in a furnace at 1000° C. This was done under the flow of a mixture of methane and hydrogen gas at a flow rate of 250 L/min. Hydrogen at a flow rate of 100 L/min was used for cooling and for opening the tube ends. The resulting black soot (as prepared double wall carbon nanotubes) was collected from the quartz tube. The amount of product obtained per run was about 2 g and carbon nanotubes were confirmed to have been successfully synthesized through various characterization techniques such as the SEM, TEM, TGA, Raman and FT-IR spectroscopy. The sample analysis was as follows:

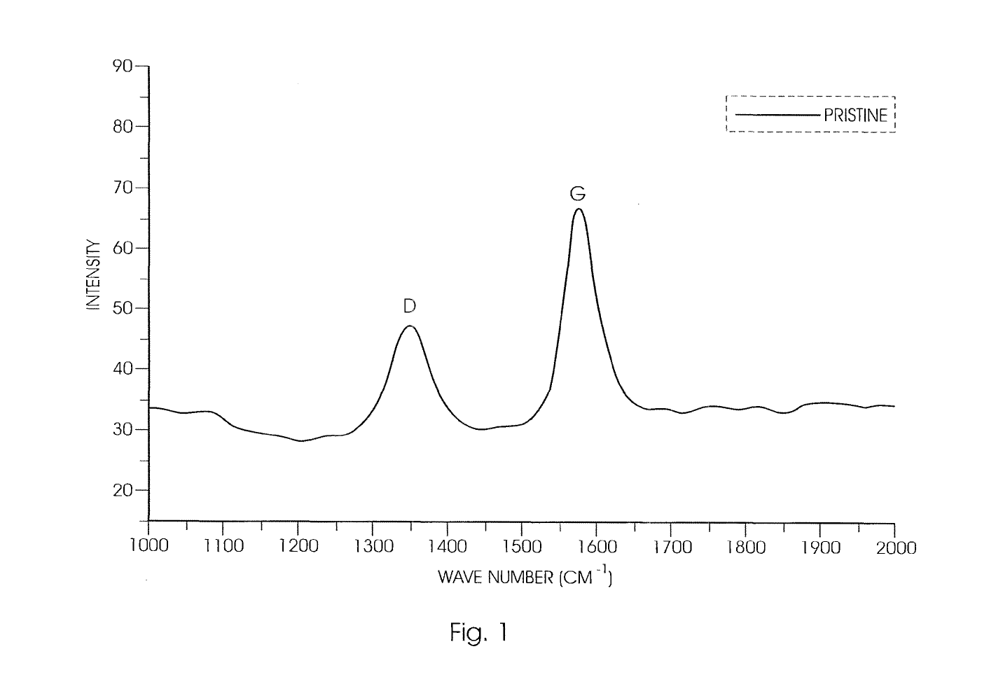

With reference to FIG. 1, the Raman spectra of pristine double walled carbon nanotubes (DWCNT) synthesis show two bands. The disorder (D mode) and tangential (G or TM mode) are bands that are characteristic of either single, double or multi walled carbon nanotubes (MWCNT). Unlike the single walled carbon nanotubes that may or may not exhibit the D mode, the Raman spectra for DWCNT and MWCNT is expected to show the disorder band at a range of 1330 cm⁻¹ and 1360 cm⁻¹. This is as a result of the larger number of defects that are spread on the several graphene sheets that make up the multi walled carbon nanotubes.

Successful synthesis of the double walled carbon nanotubes was therefore confirmed by the D mode appearing at 1350 cm⁻¹ in FIG. 1. The range for the G mode is supposed to be at around 1500 cm⁻¹ and 1600 cm⁻¹. The Raman spectra in FIG. 1 showed the G mode at 1578 cm⁻¹, a further confirmation of the crystallinity of carbon nanotubes.

I_{D}/I_{G} ratio which indicates the quality of carbon nanotubes has been calculated to be 0.67 for the pristine double walled carbon nanotubes. This indicates that there was amorphous carbon or disordered carbon and defects on the sidewalls of the tubes as was observed with the TEM (not shown).

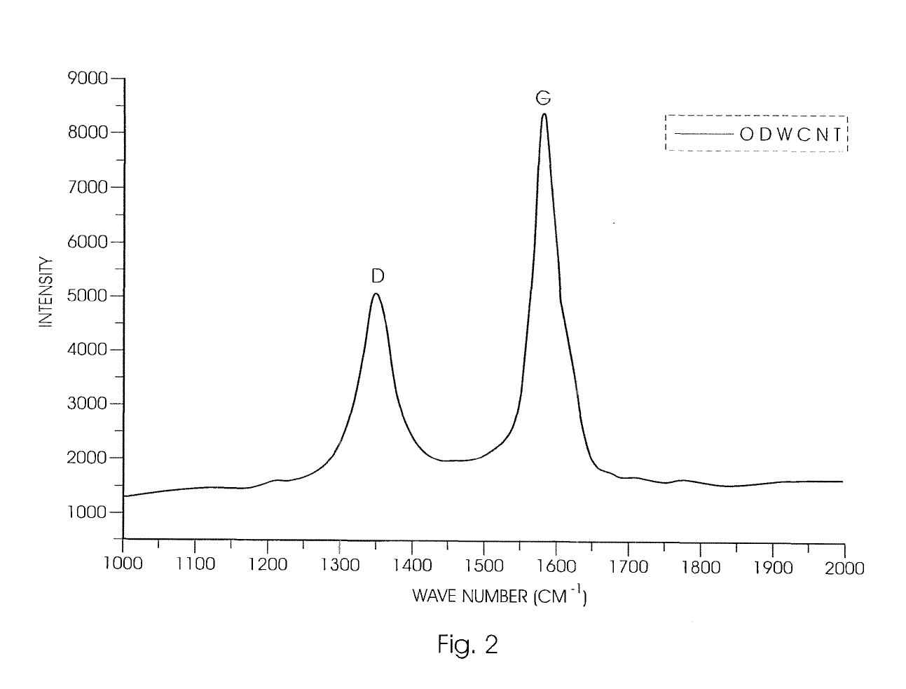

About 2 g of the residue after filtration of the pristine carbon nanotubes was refluxed in 200 ml nitric acid (55 vol %) for 3 hours at 55° C. The yield was 78% (mass:mass). Changes in the composition of the double walled carbon nanotubes due to oxidation were confirmed by the change in the intensities of the D and G bands (FIG. 2). The D band decreased in correlation with oxidation. The results are however contrary to an increase which is expected of carbon nanotubes that have been oxidised. The use of mild oxidation conditions, that is, refluxing carbon nanotubes in nitric acid for three hours instead of using sulphuric acid and nitric acid mixture could have resulted in fewer defects being introduced onto the carbon nanotube walls. The purification step ensured that most amorphous carbon and disordered carbon are removed. The I_{D}/I_{G} band therefore decreased to 0.61. There was also a slight upshift of the Raman spectra towards the right which further confirms successful oxidation.

### Example 5

### Synthesis of Covalently Bonded Bisphosphonate Compounded or Phosphorylated Double Wall Carbon Nanotubes

To introduce the bisphosphonates onto the oxidised carbon nanotubes about 3.10 g of oxidized carbon nanotubes were covalently bonded onto bisphosphonates by reacting the carboxylic acid attached on the walls of the oxidised carbon nanotubes with 3.67 g phosphoric acid and 4.38 ml phosphorus trichloride as in Scheme 5. MSA (10 ml) was used as a solvent.

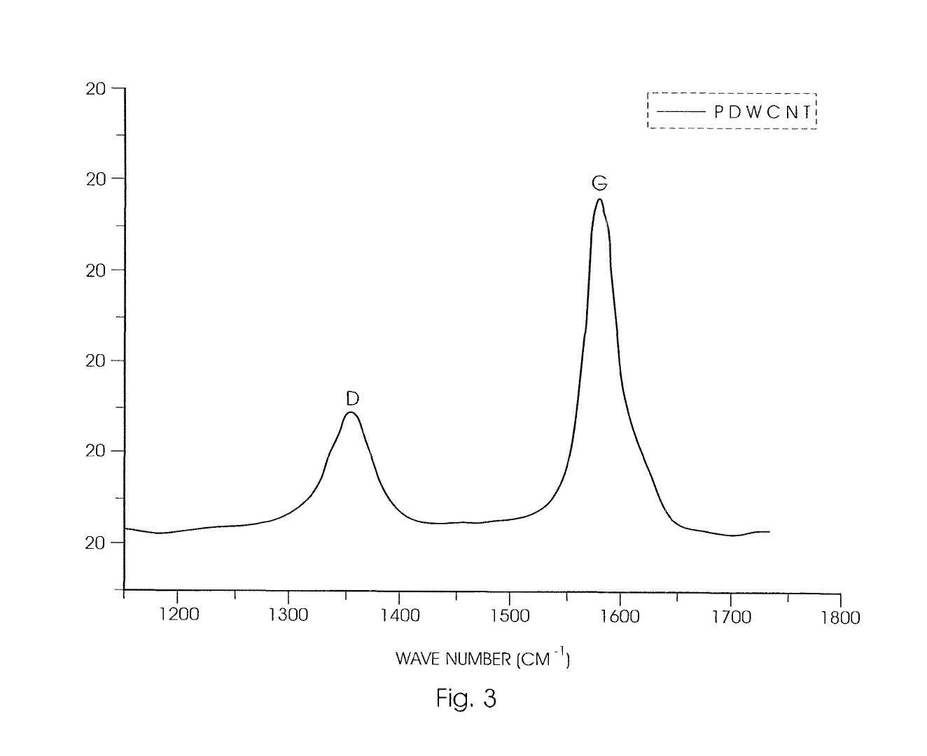

The G band in the Raman spectra for phosphorylated double walled carbon nanotubes decreased when compared with the G band of oxidised carbon nanotubes (FIG. 3). This was a proof that phosphorylation had occurred. The I_{D}/I_{G} ratio for phosphorylated carbon nanotubes was recorded to be 0.493. A slight shift of the D and G bands towards the right (1354 cm⁻¹ and 1580 cm⁻¹) further confirmed successful phosphorylation. Also, in the IR spectrum, the appearance of absorption peaks at 1200 cm⁻¹ and 1120 cm⁻¹ corresponding to P═O and P—O bonds respectively, is another proof that phosphorylation has occurred.

### Example 6

### Synthesis of ³²P Labelled Covalently Bonded Bisphosphonate Compounded Carbon Nanotubes

³²P labelled covalently bonded bisphosphonate carbon nanotubes were synthesized by performing the reaction of Scheme 5 with 20 μL ³²P labelled H₃PO₄ into a 10 mL vial. Drying of H₃PO₄ was achieved by blowing argon over the vial for 30 minutes. As a solvent, 100 μL MSA 0.023 g oxidized carbon nanotubes and 44 μL PCl₃ were added in the reaction mixture which was left to reflux for 20 hrs at 65° C. 375 μL cold water was added and the mixture was left for reflux at 65° C. for 5 hours. The pH of the mixture was adjusted by adding 120 μL NaOH to pH 4.0.

Yield: 3.2% of the ³²P labelled phosphoric acid reacted and was found to adhere to the nanotubes.

Double walled carbon nanotubes synthesis was successful as proven by the characterization techniques used. Using the SEM and the TEM it was possible to see some tubular structures that were indicative of carbon nanotubes. The Infrared spectra also showed some peaks that are characteristic of carbon nanotubes that include 1632 cm⁻¹ that correspond to a C═C bond. Carbonyl peaks at 1721 cm⁻¹, 1385 cm⁻¹ and 1067 cm⁻¹ confirmed oxidation had occurred. Additional peaks at around 1200 cm⁻¹ and 1049 cm⁻¹ were indicative of P═O and P—O respectively. Raman shifts for the D and G band also corresponded well with the known values for carbon nanotubes. A TGA plot for pristine carbon nanotubes showed a maximum weight loss at 628° C. which is characteristic of carbon nanotubes. Additional weight losses observed at confirmed successful functionalization of the carbon nanotubes. The XPS spectra confirmed the successful bonding of bisphosphonates on the carbon nanotubes. Radiolabelling of covalently bonded bisphosphonate carbon nanotubes was a success as proven by the radioactivity that was recorded by the LSC; 91% and 3.2% for ³²P hydroxy heptyl bisphosphonate and ³²P covalently bonded bisphosphonate carbon nanotubes respectively.

1. A method for producing a bisphosphonate, the method including the steps of:
providing a compound or nanostructure having carboxylic acid functional group/s; and
reacting the compound or nanostructure with phosphoric acid and a chlorinating agent, in an organic solvent.

2. The method as claimed in claim 1, wherein the bisphosphonate is a radiolabelled bisphosphonate having incorporated therein a radioisotope selected from ³²P or ³³P, and the phosphoric acid contains a radioisotope selected from ³²P or ³³P.

3. The method as claimed in claim 2, wherein the radioisotope is ³³P.

4. The method as claimed in any one of claims 1 to 3, wherein the chlorinating agent is phosphorous trichloride, phosphorous pentachloride, or oxychloride.

5. The method as claimed in claim 4, wherein the chlorinating agent is thionyl chloride (SOCl₂) or phosphorous oxy trichloride (POCl₃).

6. The method as claimed in claim 5, wherein the chlorinating agent is phosphorous trichloride

7. The method as claimed in any one of claims 1 to 6, wherein the organic solvent is methane sulphonic acid.

8. The method as claimed in any one of claims 1 to 7, wherein hypophosphorous acid (H₃PO₂) and/or ethanedinitrile (cyanogen—C₂N₂) is/are added.

9. The method as claimed in any one of claims 1 to 8, wherein the compound is a carboxylic acid selected from single chained or branched hydrocarbons.

10. The method as claimed in claim 9, wherein the carboxylic acid contains amine groups.

11. The method as claimed in claim 10, wherein the carboxylic acid is amino-propanoic acid.

12. The method as claimed in any one of claims 1 to 8, wherein the carbon nanostructure which exhibits carboxylic acid functional group/s is produced by applying defect site chemistry to covalently bond carboxylic acid functional group/s to carbon nanostructures, wherein defects on the carbon nanostructures are induced by oxidation with a strong acid.

13. The method as claimed in claim 12, wherein the acid is nitric acid.

14. A bisphosphonate having incorporated therein a radioisotope selected from ³²P or ³³P, wherein the bisphosphonate is covalently bonded to a nanostructure directly or by way of a linker.

15. The bisphosphonate covalently bonded to a nanostructure as claimed in claim 14, wherein the radioisotope is ³³P.

16. The bisphosphonate covalently bonded to a nanostructure as claimed in claim 14 or 15, wherein the nanostructure has a molecular weight of greater than about 40 kDa and less than about 400 kDa.

17. The bisphosphonate covalently bonded to a nanostructure as claimed in claim 16, wherein the nanostructure has a molecular weight of greater than about 40 kDa to about 120 kDa.

18. The bisphosphonate covalently bonded to a nanostructure as claimed in claim 17, wherein the nanostructure has a molecular weight of greater than about 60 kDa to about 100 kDa.

19. The bisphosphonate covalently bonded to a nanostructure as claimed in claim 18, wherein the nanostructure has a molecular weight of about 80 kDa

20. The bisphosphonate covalently bonded to a nanostructure as claimed in claim 14 or 15, wherein the nanostructure has a nominal diameter or principle dimension between about 5 nm to about 500 nm.

21. The bisphosphonate covalently bonded to a nanostructure as claimed in claim 20, wherein the nanostructure has a nominal diameter or principle dimension between about 20 nm to 120 nm.

22. The bisphosphonate covalently bonded to a nanostructure as claimed in claim 21, wherein the nanostructure has a nominal diameter or principle dimension between about 50 nm to 100 nm.

23. The bisphosphonate covalently bonded to a nanostructure as claimed in any one of the claims 14 to 22, wherein the bisphosphonate has the general structure: 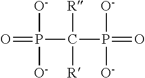
where:
R′ is hydrogen, alkyl containing from 1 to about 20 carbon atoms, alkenyl containing from 2 to about 20 carbon atoms, aryl, phenylethenyl, benzyl, halogen, hydroxyl, amino, substituted amino, —CH₂COOH, —CH₂PO₃H₂, —CH(PO₃H₂)(OH), or —CH₂C(PO₃H₂)₂ₙ—H where n is 1 to 15; and
R″ is a nanostructure covalently bonded directly or via a linker to the bisphosphonate.

24. The bisphosphonate covalently bonded to a nanostructure as claimed in claim 23, wherein the linker is alkyl containing from 1 to about 10 carbon atoms, alkenyl containing from 2 to about 10 carbon atoms, amino or substituted amino.

25. The bisphosphonate covalently bonded to a nanostructure as claimed in claim 23 or 24, wherein R′ is hydroxyl.

26. The bisphosphonate covalently bonded to a nanostructure as claimed in any one of claims 14 to 25, wherein the nanostructure is a carbon nanostructure.

27. The bisphosphonate covalently bonded to a nanostructure as claimed in claim 26, wherein the carbon nanostructure is a carbon nanotube.

28. The bisphosphonate covalently bonded to a nanostructure as claimed in claim 27, wherein the carbon nanotube is single-walled or multi-walled.

29. The bisphosphonate covalently bonded to a nanostructure as claimed in claim 28, wherein the carbon nanotube is single-walled.

30. The bisphosphonate covalently bonded to a nanostructure as claimed in claim 29, wherein the single-walled nanotube has a diameter of 0.4 to 5 nm.

31. The bisphosphonate covalently bonded to a nanostructure as claimed in claim 30, wherein the single-walled nanotube has a diameter of 0.5 to 1 nm.

32. The bisphosphonate covalently bonded to a nanostructure as claimed in claim 31, wherein the carbon nanotube is multi-walled.

33. The bisphosphonate covalently bonded to a nanostructure as claimed in claim 32, wherein the carbon nanotube is double-walled.

34. The bisphosphonate covalently bonded to a nanostructure as claimed in claim 33, wherein the double-walled carbon nanotube has a diameter of 4.5 to 100 nm.

35. The bisphosphonate covalently bonded to a nanostructure as claimed in claim 34, wherein the double-walled carbon nanotube has a diameter of about 5.0 nm.

36. The bisphosphonate covalently bonded to a nanostructure as claimed in claim 35, wherein the carbon nanostructure is a carbon nanosphere.

37. The bisphosphonate covalently bonded to a nanostructure as claimed in claim 36, wherein the carbon nanosphere has a diameter of about 5 to 20 nm.

38. The bisphosphonate covalently bonded to a nanostructure as claimed in claim 37, wherein the carbon nanosphere has a diameter of about 12 nm.

39. The bisphosphonate covalently bonded to a nanostructure as claimed in claim 38, wherein the carbon nanosphere has a molecular weight of about 50 to 100 kDa.

40. The bisphosphonate covalently bonded to a nanostructure as claimed in claim 39, wherein the carbon nanosphere has a molecular weight of about 80 kDa.

41. A method of treating calcific tumours in a patient, the method including the step of administering to the patient a radiolabelled bisphosphonate which is covalently bonded to a nanostructure directly or through a linker, as defined in any one of claims 14 to 40.

42. A radiolabelled bisphosphonate which is covalently bonded to a nanostructure directly or through a linker, as defined in any one of claims 14 to 40, for use in the treatment of calcific tumours in a patient.

43. The use of a radiolabelled bisphosphonate which is covalently bonded to a nanostructure directly or through a linker, as defined in any one of claims 14 to 40, in a method of manufacturing a medicament or use in the treatment of calcific tumours in a patient.
